(19) United States
(12) Patent Application Publication
(10) Pub. No.: US 2013/0327086 A1
(43) Pub. Date: Dec. 12, 2013

(54) METHOD FOR THE SIMPLIFIED REMOVAL OF A REACTION PRODUCT FROM REACTION GAS MIXTURES USING AT LEAST TWO-FOLD PARTIAL CONDENSATION

(71) Applicants: Amgad Salah Moussa, Koln (DE); Peter Lehner, Baytown, TX (US); Knut Sommer, Krefeld (DE)

(72) Inventors: Amgad Salah Moussa, Koln (DE); Peter Lehner, Baytown, TX (US); Knut Sommer, Krefeld (DE)

(73) Assignee: BAYER INTELLECTUAL PROPERTY GmbH, Monheim (DE)

(21) Appl. No.: 13/878,534

(22) PCT Filed: Oct. 17, 2011
(86) PCT No.: PCT/EP2011/068127

§ 371 Date: Aug. 30, 2013

(30) Foreign Application Priority Data
Oct. 21, 2010 (DE) 10 2010 042 760.8

(51) Int. Cl. C07C 7/09

(52) U.S. Cl. CPC C07C 7/09
USPC 62/617

(57) ABSTRACT

The present invention relates to a process for simplified separation of a reaction product (P) from reaction gas mixtures by means of partial condensation of the reaction gas mixture consisting of the reaction product (P), at least one high boiler (H) and at least one low boiler (L).

The present invention relates to a process for simplified separation of a reaction product (P) from reaction gas mixtures by means of at least two partial condensations of the reaction gas mixture consisting of the reaction product (P), at least one high boiler (H) and at least one low boiler (L), and uncondensable reaction gases (G).

Chemical process technology often involves execution of gas phase reactions, in the course of which a reactant gas or reactant gas mixture is converted, optionally in the presence of a catalyst, to a very substantially pure gaseous reaction product.

Especially in large chemical processing plants, however, it is often the case that the aforementioned gas phase reaction is not run with a quantitative conversion of all reactants, which is often for economic reasons in particular, since such a quantitative conversion would usually be associated with a significant decrease in the space-time yield. In other cases, however, this is not technically possible merely due to the fact that the reaction is equilibrium-limited. In many other cases, in the course of the main reaction to give the desired reaction product, side reactions also form further reaction gases as secondary components, which subsequently have to be removed. Such further reaction gases in the form of secondary components then also constitute high boilers (H) and/or low boilers (L).

The direct consequence of the aforementioned facts is that, at the end of the aforementioned gas phase reaction, a reaction gas mixture is obtained and then has to be sent to a removal of reaction gases present alongside the desired gaseous reaction product(s), in order finally to obtain a reaction product of sufficient purity.

One example of such processes is that of processes for preparing aniline. Aniline is an important intermediate on the route to production of polymers, such as polyurethanes. It is prepared on the industrial scale generally by catalytic gas phase hydrogenation of nitrobenzene.

Such an industrial scale gas phase process forms not only the desired gaseous aniline reaction product, but also the reaction gas ammonia as a by-product. In any case, however, such a gas phase reaction is not executed in such a way that a quantitative conversion is achieved, and so, at the end of the gas phase reaction, a reaction gas mixture is obtained comprising the desired gaseous aniline reaction product, and also the reaction gases ammonia, nitrobenzene, the excess hydrogen, and further gaseous impurities introduced, for instance, through the nitrobenzene and hydrogen reactant gases, for instance inert gases (e.g. nitrogen, carbon dioxide and water vapour). In addition, the aforementioned reaction also forms diphenylamine as a high boiler (H) through side reactions (addition reaction).

The reaction gases present in the reaction gas mixture alongside the gaseous reaction product (P) desired, as obtained from the aforementioned processes or else other similar industrial scale gas phase reactions, can be roughly divided into the groups of the “low boilers” (L), i.e. compounds or azeotropically boiling mixtures of individual compounds having boiling points below that of the desired reaction product at ambient pressure (1013 hPa), and of the “high boilers” (H), i.e. compounds or azeotropically boiling mixtures of individual compounds having boiling points above that of the desired reaction product at ambient pressure (1013 hPa). In this context, the reaction product (P) which is to be removed by the invention described hereinafter is not necessarily identical to the desired reaction product of the reaction executed prior to the removal described here, but may also be a reactant, by-product or even a mixture of reactant and desired reaction product and/or by-product of the reaction in question.

Whether the reaction gases belong to one group or the other is decided in the specific case by the relationship between the respective boiling point of the desired reaction product (P) and the boiling point of the further reaction gas in each case.

Using the example of the aforementioned preparation of aniline from nitrobenzene by means of catalytic gas phase hydrogenation, nitrobenzene with a boiling point of 211° C. at ambient pressure (1013 hPa) is a high boiler (H) compared to the aniline reaction product (P), since this has a boiling point of 184° C. at ambient pressure (1013 hPa). Diphenylamine too, which is formed in a side reaction, should be regarded here as a high boiler (H; boiling point 302° C. here at ambient pressure of 1013 hPa).

The above terms “high boilers” (H) and “low boilers” (L) are used hereinafter according to the definition above. Unless explicitly stated otherwise below, the above definition is to be applied to these terms in the context of the present invention.

A subgroup of the low boilers (L) which should be viewed separately therefrom in the context of the present invention is that of the “uncondensable reaction gases” (G). Uncondensable reaction gases (G) are understood in the context of the present invention to mean those low boilers (L) having a boiling point below room temperature (23° C.) at ambient pressure (1013 hPa).

Typical representatives of the low boilers (L) included in the group of the uncondensable reaction gases according to the present invention are, for instance, the “inert gases”, as commonly known to those skilled in the art by this name, i.e. gases such as, for instance, noble gases (argon, helium, etc.), carbon dioxide, nitrogen. The group of the uncondensable reaction gases likewise includes, for example, hydrogen, which is added in excess to the preceding reaction, for instance in the context of hydrogenation reactions, and carbon monoxide. Depending on the composition of the reaction gas mixture, these are regarded collectively hereinafter, possibly together with further reaction gases, as uncondensable reaction gases (G).

Reaction gases in connection with the present invention denote all compounds other than the reaction product (P) supplied in gaseous form from the gas phase reaction to the process according to the invention. Reaction gases thus include gaseous impurities, inert gases, uncondensable reaction gases (G), low boilers (L) and high boilers (H).

Reactant gases or reactant gas mixtures in the context of the present invention denote gaseous compounds which are supplied to the aforementioned gas phase reaction. Such reactant gases or reactant gas mixtures leave the gas phase reaction either as reaction gases or are chemically converted thereto or are chemically converted to the reaction product (P).

Reaction gas mixture according to the present invention denotes a mixture consisting of reaction product and reaction gases.

In order to separate the reaction product (P) in the reaction gas mixture from the reaction gases comprising aforementioned high boilers (H), low boilers and uncondensable reaction gases (G), in practical process technology, the commonly known technique of rectification or distillation is nearly always employed.

Rectification and distillation are each based on the basic physical principle that a substance mixture, at a particular pressure and at a particular temperature, has a different composition in its gaseous component from that in its liquid component. This is generally exploited by condensing and vaporizing such reaction gas mixtures, which, in practical process technology, is effected several times in succession in what are called rectification or distillation columns. The quality and the expenditure associated with the separation of the substances that constitute the reaction gases from the reaction product (P) is determined here not least by the separation of the respective boiling points of the substances from one another at the respective pressure. The further these are apart from one another, i.e. the further the boiling points of all low boilers (L) and high boilers (H) are removed from the boiling point of the reaction product (P), the simpler and better the separation of the reaction product (P).

As just explained, the rectificative or distillative separation is based on the condensing and vaporizing of the reaction gas mixture present completely in gaseous form at the end of the gas phase reaction.

Accordingly, the reaction gas mixture, in the customarily operated processes for separating the reaction product (P), in which the reaction gas mixture is fed in its entirety to a rectification or distillation column, has to be repeatedly condensed and vaporized.

In the case of reaction gas mixtures in which the boiling points of reaction product (P) and reaction gases are close to one another, it follows that the overall reaction gas mixture, for a good separation and hence purity of the reaction product (P), has to be condensed and vaporized with corresponding frequency in a rectification column with reflux.

If the reaction gas mixture consists of reaction product (P) and a high proportion of reaction gases, a large proportion of which in turn are high boilers (H), the energy expenditure which has to be expended for the separation is considerable.

However, it is generally always the case that a large amount of energy has to be expended for the aforementioned separation.

The known prior art addresses this problem area with a multitude of proposed industrial solutions. For example, there are proposals of solutions which, with the aid of further reactions, convert the compounds in the reaction gases of the initially fully condensed reaction gas mixture partly to compounds which can no longer be vaporized under the conditions of the later distillation or rectification and can thus be separated in a simplified manner. In other words, these reaction gases are converted chemically to very particularly high-boiling high boilers (H).

For instance, using the example of aromatic amines as the reaction product (P), EP 2 028 176 A1 discloses a process in which the reaction gas mixture is first fully condensed, the full condensate is separated from the water which automatically separates out through phase separation up to the solubility concentration of the aromatic amine therein, and then the “crude” aromatic amine thus obtained is reacted with an aqueous solution of an alkali metal hydroxide, as a result of which the reaction gases in the form of compounds having phenolic hydroxyl groups are substantially converted to the corresponding alkali metal salts.

These alkali metal salts in the process disclosed therein are so high-boiling that they subsequently accumulate in the bottom of the rectification column and essentially do not get into the other parts of the rectification column, which means that an energy saving arises from the lack of vaporization and condensation thereof.

However, the process disclosed therein additionally requires that the entire reaction gas mixture be condensed and heated together with the additional aqueous solution of an alkali metal hydroxide but without the water removed beforehand. This saves energy expenditure, but at the same time requires additional energy expenditure for heating of the aqueous solution of an alkali metal hydroxide and additionally complicates the process through the requirement for addition of a further feedstock.

As well as the aforementioned alternative limited to application to processes for separation of aromatic amines, there exist, as already stated, a multitude of further proposed solutions in the prior art. A feature common to these is that achievement of a simplified separation always involves addition of more or less complex additional reaction steps and/or other separation steps, such as extraction, membrane processes etc.

A similar process which, however, dispenses with the need for further reaction of reaction gases is described in U.S. Pat. No. 6,388,155 B1.

The process described therein relates to the preparation and purification of the styrene reaction product which is obtained by the dehydrogenation of ethylbenzene. In the process, the reaction gas mixture is first cooled but not condensed, and fed to a scrubber under reflux. The cooling in the process according to U.S. Pat. No. 6,388,155 B1 serves particularly for energy recovery from the reaction gas mixture. The gas stream leaving the aforementioned scrubber is fed to the sole condenser in the process, in which the gases are partly condensed. The condensed component of the gases is fed to a phase separation. A portion of the liquids is subsequently subjected to a distillation. The scrubber according to U.S. Pat. No. 6,388,155 B1 may be any desired gas/liquid contact apparatus. Overall, the combination of scrubber, condenser and phase separator can be regarded as a unit for execution of a partial condensation, this partial condensation being executed by reflux in more than one theoretical plate. According to a variant likewise described in U.S. Pat. No. 6,388,155 B1, a vaporizer is added at the bottom of the aforementioned scrubber, which means that the scrubber can be regarded no longer as a scrubber but as a particular form of rectification column, since both a condensation of the top stream and recycling of the condensate thus obtained into the scrubber, and also vaporization of the bottom stream and recycling of the vapour into the scrubber, are envisaged. In the scrubber, there is then phase contact between vapour and condensate and, depending on the two temperatures thereof, establishment of one or more new gas/liquid equilibria in which reaction gases and reaction product accumulate in various proportions depending on the temperature. This is the complete principle of function of a rectification column described above.

According to the variants described in U.S. Pat. No. 6,388,155 B1, the reaction step described, for instance, in EP 2 028 176 A1 is replaced either by the aforementioned partial, multistage condensation step or a further rectification.

Overall, however, U.S. Pat. No. 6,388,155 B1 does not disclose that it would be possible to execute a partial condensation (whether one-stage or multistage) more than once. More particularly, in the light of the proposed replacement by a rectification step, this is not suggested as being advantageous.

Accordingly, in U.S. Pat. No. 6,388,155 B1 too, either the entire reaction gas mixture is again treated in a multiple vaporization and condensation of the entire reaction gas mixture in the case of the particular embodiment as a rectification step, which entails the energy disadvantages already described, or optimal exploitation of the enthalpy of the reaction gas mixture is not achieved in the case of single execution of partial multistage condensation, as a result of which it would have been possible to dispense with at least one of the distillation columns described in this case according to U.S. Pat. No. 6,388,155 B 1 with corresponding saving of energy. In the distillation columns, the aforementioned disadvantageous full condensation and vaporization of all of the components of the reaction gas mixture supplied is in turn effected.

It follows that, in the process according to U.S. Pat. No. 6,388,155 B1, the energy expenditure of the last rectifications disclosed is reduced, but the energy expenditure is still not minimized and hence not optimized with full exploitation of the enthalpy of the reaction gas mixture.

Accordingly, none of the prior art publications cited addresses how it is possible to achieve reduction in the proportion of the reaction gas mixture which has to be vaporized and condensed again in a later separation of the reaction product in such a way that the energy expenditure for the overall process can likewise be reduced while maintaining a good separation of the reaction product with a simple apparatus design. More particularly, in all such prior art processes, proportions of the enthalpy of the reaction gas mixture are always wasted through unnecessary condensation and vaporization.

Proceeding from the prior art, it is thus an object of the present invention to provide a process for separating a reaction product (P) from gas phase reactions, in which the energy expenditure for the overall process can be reduced with maintenance of a good separation of the reaction product with a simple apparatus design. More particularly, optimal exploitation of the enthalpy of the reaction gas mixture is to be achieved here, by avoiding unnecessary condensation and vaporization.

It has now been found that, surprisingly, the aforementioned object is achieved by a process for simplified separation of a reaction product (P) from reaction gas mixtures at a temperature above room temperature (23° C.), consisting of the aforementioned reaction product (P), at least one low boiler (L) and/or uncondensable reaction gas (G) and at least one high boiler (H), comprising at least one distillation and/or rectification step, characterized in that the at least one distillation and/or rectification step is preceded by subjecting the reaction gas mixture to at least two partial condensation steps in which, in at least one
(a) first partial condensation step, at least 80% by weight of the high boilers (H) supplied to this first partial condensation step are condensed, and at least one liquid stream comprising high boilers (H) and any reaction product (P), and a gas stream comprising the reaction product (P), are thus obtained therefrom, and, in a
(b) second partial condensation step, at most 5% by weight of the low boilers (L) supplied to this second partial condensation step and/or uncondensable reaction gases (G) are condensed, and at least one liquid stream comprising the supplied reaction product (P), and a gas stream comprising the supplied low boilers (L) and/or uncondensable reaction gases (G), are thus obtained therefrom,
   by
   1) supplying at least one of the liquid streams comprising reaction product (P) to the at least one distillation and/or rectification step at a point in the distillation and/or rectification column above the point from which the reaction product (P) is drawn off from this distillation and/or rectification column, and/or
   2) supplying at least one of the liquid streams comprising high boilers (H) to the at least one distillation and/or rectification step at a point in the distillation and/or rectification column below the point from which the reaction product (P) is drawn off from this distillation and/or rectification column.

The above-described process according to the invention in its simplest embodiment makes it possible for the first time to execute a preliminary fractionation before the actual separation in the distillation or rectification, such that the stream comprising the reaction product (P) obtained from steps a) and b) of the process is reduced and hence the energy to be expended for the distillative or rectificative separation is significantly reduced solely thereby.

The aforementioned partial condensations executed in accordance with the invention can be executed in simple apparatuses commonly known to those skilled in the art. Theoretically, it is sufficient for this purpose to provide cooled vessels, or even uncooled vessels in special cases where the reaction product (P) has a boiling point in the region of ambient temperature (23° C.) or higher, with an inlet for the reaction gas mixture, an outlet for the condensate and an outlet for the remaining gas stream. Typically, however, condensation heat exchangers commonly known to those skilled in the art will certainly be used to execute the inventive partial condensations. In the preferred embodiments of the process according to the invention described later, preferred variations of such apparatuses are additionally explained.

Effectively, it is possible for the person skilled in the art in a simple manner, proceeding from a reaction gas mixture known in terms of its composition and further parameters, such as pressure and temperature, to execute the partial condensations described in a) or b) such that the weight ratios specified are attained. More particularly, the person skilled in the art is aware from the relevant literature of the phase equilibrium curves of the individual constituents of the reaction gas mixtures and can thus select the crucial temperature in the partial condensation steps such that the abovementioned ratios are attained.

In the preferred embodiments of the invention described later, this is shown once again for preferred processes, and this is demonstrated once again for selected reaction gas mixtures in the Working Examples.

The requirement for supply of the various above-described liquid streams into the at least one distillation and/or rectification column at particular points ensures that, as well as the aforementioned advantages of energy saving before the distillation and/or rectification, the energy advantage is also maintained in the later distillation and/or rectification.

In a first preferred development of the present invention, the first partial condensation according to step a) and the second partial condensation according to step b) of the process according to the invention are executed in direct succession, the gas stream from the first partial condensation being supplied immediately to the second partial condensation.

Within this first preferred development, it is preferable when the gas stream obtained from the second partial condensation, also comprising reaction product (P), is sent to a third partial condensation in which at least 80% by weight of the reaction product (P) supplied to the third partial condensation is condensed.

This third partial condensation is especially advantageous when the gas stream from the second partial condensation, as well as the low boilers (L) and/or uncondensable reaction gases (G) supplied thereto, also comprises significant proportions of the reaction product (P), for instance because the phase equilibrium of the remaining reaction gas mixtures at the end of the second partial condensation is unfavourable, or when the low boilers (L) and/or uncondensable reaction gases (G) are also to be separated from one another in the process according to the invention.

In a second preferred development of the present invention, at least one of the partial condensations is executed with reflux of the condensate of this partial condensation.

Preference is given here to diverting some or all of the recycled condensate and conducting it out of this partial condensation as a further liquid stream.

According to the reflux ratio and/or proportion of the condensate diverted, a different composition of the one or two liquid streams which are then conducted out of the partial condensation is the result.

An execution under reflux with aforementioned diversion of a further liquid stream is advantageous especially when the gas stream supplied to such a partial condensation comprises low boilers (L), uncondensable reaction gases (G) and reaction product (P), or high boilers (H), reaction product (P) and low boilers (L), since such an execution under reflux with diversion also enables the simultaneous separation of low boilers (L) and reaction product (P) or high boilers (H) and reaction product (P) into two different liquid streams, and uncondensable reaction gases (G) or low boilers (L) into a remaining gas stream.

Apparatuses which allow such a partial condensation under reflux are the above-described condensation heat exchangers with upstream scrubbing column and downstream collecting vessel. A diversion is typically implemented by the provision of an outlet with a control valve from the return line of the condensate into the scrubbing column.

In a third preferred development of the present invention, at least one of the partial condensations also comprises a phase separation between two liquid phases which are not completely miscible with one another under the conditions of the partial condensation, such that the partial condensation according to this preferred development, just as in the case of the above-described second preferred development with diversion, gives two liquid streams and one gas stream.

This allows the constituents of the reaction gas mixture which may already have been pre-fractionated by the at least one partial condensation to be fractionated once again without further energy expenditure.

Typically, these two liquid streams are then a first liquid stream comprising essentially nonpolar constituents of the partly condensed reaction gas mixture and a second liquid stream comprising essentially polar constituents of the partly condensed reaction gas mixture.

The partial condensation according to this third preferred development can also be combined with the partial condensation according to the second preferred development and preferred embodiments thereof with diversion. It may thus be possible to obtain up to three liquid streams of different composition from one partial condensation step.

The apparatuses needed for execution of the process according to this third preferred development are common knowledge to those skilled in the art. The same applies in the case of combination with the partial condensation according to the second preferred development. A non-exclusive example of an apparatus for execution of the partial condensation according to the third preferred development is, for instance, a condensation heat exchanger with connected calming vessel, from which two outlets for liquids lead out, one at the base of this vessel and one above the phase boundary of the liquids in the vessel, as does an outlet for the remaining reaction gases at the top of the vessel.

In the same way as the second and the third preferred developments of the present invention can be combined with one another, the first preferred development with its preferred embodiments can also be combined with these.

According to a first especially preferred development of the present invention, the first partial condensation according to step b) of the invention is accordingly executed directly after a partial condensation according to step a) of the invention, and step b) is followed by execution of a partial condensation with reflux of the condensate of this partial condensation, a portion of this recycled condensate also being diverted and conducted out of this third partial condensation as a further liquid stream.

According to a second especially preferred development of the present invention, the first partial condensation according to step b) of the invention is executed directly after a partial condensation according to step a) of the invention, and this first partial condensation is executed with reflux of the condensate of this partial condensation, a portion of this recycled condensate also being diverted and conducted out of this first condensation as a further liquid stream, while the second partial condensation according to step b) also includes a phase separation between two liquid phases which are not completely miscible with one another under the conditions of the partial condensation, such that a liquid stream comprising essentially polar constituents of the partly condensed reaction gases and a liquid stream comprising essentially nonpolar constituents of the partly condensed reaction gases are conducted out of this second partial condensation.

According to a third especially preferred development of the present invention, the first partial condensation according to step b) of the invention is executed directly after a partial condensation according to step a) of the invention, and this first partial condensation is executed with reflux of the condensate of this partial condensation, a portion of this recycled condensate also being diverted and conducted out of this first condensation as a further liquid stream, while the second partial condensation according to step b) is likewise executed with reflux of the condensate of this second partial condensation, and a portion of this recycled condensate is also diverted and conducted out of this second partial condensation as a further liquid stream.

According to a fourth especially preferred development of the present invention, the first partial condensation according to step b) of the invention is executed directly after a partial condensation according to step a) of the invention, and this first partial condensation is executed with complete reflux of the condensate of this partial condensation and with phase separation between two liquid phases which are not completely miscible with one another under the conditions of the partial condensation, such that a further liquid stream is likewise conducted out of this first condensation, while the second partial condensation according to step b) encompasses only a phase separation between two liquid phases which are not completely miscible with one another under the conditions of the partial condensation, such that a liquid stream comprising essentially polar constituents of the partly condensed reaction gases and a liquid stream comprising essentially nonpolar constituents of the partly condensed reaction gases are conducted out of this second partial condensation.

Within the first especially preferred development of the process according to the invention just described, it is especially preferable that the reaction gas mixture comprises the reaction product (P) toluenediamine, the uncondensable reaction gases (G) hydrogen and nitrogen, the low boiler (L) water and high boilers having a boiling point of more than 300° C. at ambient pressure (1013 hPa), and that this reaction gas mixture is sent to a first partial condensation, from which a first liquid stream comprising the entirety of the high boilers (H) and a proportion of not more than 10% by weight of the toluenediamine is obtained, and a first gas stream comprising all of the hydrogen and nitrogen and all of the water, and at least 90% by weight of the toluenediamine, is obtained.

In this context, it is very especially preferable when the aforementioned first gas stream is sent to the second partial condensation, from which a second gas stream comprising all of the hydrogen and nitrogen and all of the water from the first gas stream sent to the second partial condensation, as well as not more than 20% by weight of the toluenediamine supplied thereto, is obtained. In addition, a second liquid stream comprising only the toluenediamine is obtained from this second partial condensation.

The second gas stream is sent to the third partial condensation under reflux and with diversion of a third and a fourth liquid stream from the third partial condensation. A third gas stream leaves the third partial condensation, this comprising not more than 30% by weight of the water supplied thereto with the second gas stream and all of the nitrogen and hydrogen. The third liquid stream comprises the water, and the fourth liquid stream contains the toluenediamine alone.

The aforementioned first liquid stream, according to the especially preferred execution of the first preferred development described here, is fed to a first distillation column, in the middle thereof between stripping section and rectifying section, and pure toluenediamine is withdrawn from the first distillation column at the top, as are the high boilers (H) at the bottom.

The aforementioned second and any fourth liquid stream too are especially preferably fed to a second distillation column, in the rectifying section thereof, and the pure toluenediamine is withdrawn therefrom between stripping section and rectifying section, as is the water at the top.

All partial condensations according to the above-detailed especially preferred embodiment of the first especially preferred development of the invention are conducted here typically at a pressure in the range from 2 to 5 bar, preferably at a pressure of about 3 bar.

The first partial condensation is conducted here typically at a temperature in the range from 200° C. to 220° C., and the second partial condensation at a temperature in the range from 160° C. to 200° C.

The third partial condensation is preferably conducted in the region of the scrubbing column at a temperature in the range from 85° C. to 160° C., and condensation heat exchanger and downstream collecting vessel at a temperature in the range from 50° C. to 90° C.

Within the combination of the above-described first and second preferred developments of the process according to the invention, it is especially preferable that the reaction gas mixture comprises the reaction product (P) methanol, the uncondensable reaction gases (G) carbon monoxide, carbon dioxide, hydrogen, nitrogen and methane, the low boiler (L) dimethyl ether and the high boilers (H) water and ethanol, and that this reaction gas mixture is sent to a first partial condensation under reflux, from which a first liquid stream comprising the entirety of the water, a proportion of the ethanol and a proportion of not more than 30% by weight of the methanol is obtained, a second liquid stream comprising methanol and ethanol is obtained, and a first gas stream comprising all of the hydrogen and nitrogen and all of the carbon monoxide, and at least 90% by weight of the carbon dioxide, methane and dimethyl ether, is obtained.

In this context, it is very especially preferable when the aforementioned first gas stream is sent to the second partial condensation, from which a second gas stream comprising all of the hydrogen and nitrogen and all of the carbon monoxide in the first gas stream sent to the second partial condensation together with at least 95% by weight each of the carbon dioxide, methane and dimethyl ether in the first gas stream sent to the second partial condensation, as well as not more than 50% by weight of the methanol sent thereto, is obtained. In addition, it is very especially preferable when a third liquid stream comprising methanol, ethanol and not more than 5% by weight each of the dimethyl ether and carbon dioxide in the first gas stream sent to the second partial condensation is obtained from this second partial condensation.

The aforementioned first liquid stream, according to the especially preferred execution of the combination of the first and second preferred developments described here, is fed to a first distillation column, in the middle thereof between stripping section and rectifying section, and pure dimethyl ether is withdrawn from the first distillation column at the top, water and ethanol at the bottom, and methanol in the region of the stripping section.

The aforementioned second and any third liquid stream too are especially preferably fed to a second distillation column, from which are withdrawn the dimethyl ether at the top and methanol at the bottom, optionally together with remaining ethanol, or alternatively fed to what is called a stripper, as generally known to those skilled in the art, from which methanol is likewise withdrawn at the bottom, optionally together with remaining ethanol, and dimethyl ether at the top.

The two partial condensations according to the above-detailed especially preferred embodiment of the invention are conducted here typically at a pressure in the range from 50 to 90 bar, preferably at a pressure of about 75 bar.

The first partial condensation is preferably conducted in the region of the scrubbing column at a temperature in the range from 120° C. to 160° C., and condensation heat exchanger and downstream collecting vessel at a temperature in the range from 70° C. to 120° C.

The second partial condensation is typically conducted here at a temperature in the range from 40° C. to 80° C.

Within the combination of the above-described first and second preferred developments of the process according to the invention, it is additionally especially preferable that the reaction gas mixture comprises the reaction product (P) benzene, the uncondensable reaction gases (G) hydrogen, nitrogen and methane, and the high boiler (H) toluene, and that this reaction gas mixture is sent to a first partial condensation under reflux with diversion, from which a first liquid stream comprising the entirety of the toluene and a proportion of not more than 50% by weight of the benzene is obtained, a second liquid stream comprising benzene is obtained, and a first gas stream comprising all of the hydrogen and nitrogen and all of the methane, and also benzene, is obtained.

In this context, it is very especially preferable when the aforementioned first gas stream is sent to the second partial condensation, from which a second gas stream comprising all of the hydrogen and nitrogen and all of the methane from the first gas stream sent to the second partial condensation is obtained. In addition, a third liquid stream comprising at least 95% by weight of the benzene sent to the second partial condensation is especially preferably obtained from this second partial condensation.

The aforementioned first liquid stream, according to the especially preferred execution of the combination of the first and second preferred developments described here, is fed to a first distillation column, in the middle thereof between stripping section and rectifying section, and pure benzene is withdrawn from the first distillation column at the top, as is pure toluene at the bottom.

The aforementioned second and any third liquid stream too are especially preferably fed to a second distillation column, from which pure benzene is withdrawn at the top, or alternatively fed to what is called a stripper, as generally known to those skilled in the art, from which pure benzene is likewise withdrawn at the top.

The two partial condensations according to the above-detailed especially preferred embodiment of the invention are conducted here typically at a pressure in the range from 30 to 40 bar, preferably at a pressure of about 35 bar.

The first partial condensation is preferably conducted in the region of the scrubbing column at a temperature in the range from 140° C. to 220° C., and condensation heat exchanger and downstream collecting vessel at a temperature in the range from 70° C. to 150° C.

The second partial condensation is typically conducted here at a temperature in the range from 50° C. to 80° C.

Within the above-described second especially preferred development of the process according to the invention, it is additionally especially preferable that the reaction gas mixture comprises a mixture of aniline and phenol as reaction product (P), hydrogen, nitrogen and ammonia as uncondensable reaction gases (G), water and benzene as low boilers (L) and diphenylamine as high boiler (H), and that this reaction gas mixture is sent to a first partial condensation under reflux with diversion, from which a first liquid stream comprising the entirety of the diphenylamine and a proportion of not more than 50% by weight of the reaction product (P) is obtained, a second liquid stream comprising the reaction product (P) is obtained, and a first gas stream comprising all of the hydrogen and nitrogen and all of the ammonia, and also at least 95% by weight each of benzene and water, is obtained.

In this context, it is very especially preferable that the aforementioned first gas stream is sent to the second partial condensation with phase separation, from which a second gas stream comprising all of the hydrogen and nitrogen and at least 80% by weight of ammonia from the first gas stream sent to the second partial condensation is obtained. In addition, a third liquid stream comprising at least 95% by weight of the benzene sent to the second partial condensation and a fourth liquid stream comprising at least 95% by weight of the water sent to the second partial condensation are obtained from the second partial condensation.

The aforementioned first and second liquid streams are, according to the especially preferred execution of the second especially preferred development described here, sent to a distillation column. In this case, the first liquid stream is supplied in the stripping section of the column and the second liquid stream in the rectifying section of the column. Between the stripping and rectifying sections of the column, the reaction product (P) is removed.

The two partial condensations according to the above-detailed especially preferred embodiment of the invention are conducted here typically at a pressure in the range from 2 to 10 bar, preferably at a pressure of about 5 bar.

The first partial condensation is preferably conducted in the region of the scrubbing column at a temperature in the range from 140° C. to 150° C., and condensation heat exchanger and downstream collecting vessel at a temperature in the range from 110° C. to 150° C.

The second partial condensation is typically conducted here at a temperature in the range from 60° C. to 120° C.

Within the above-described third especially preferred development of the process according to the invention, it is additionally especially preferable that the reaction gas mixture comprises the reaction product (P) isopropylbenzene, the uncondensable reaction gases (G) propylene and propane, the low boiler (L) benzene and the high boiler (H) diisopropylbenzene, and when this reaction gas mixture is sent to a first partial condensation under reflux with diversion, from which a first liquid stream comprising the entirety of the diisopropylbenzene and a proportion of not more than 10% by weight of the isopropylbenzene is obtained, a second liquid stream comprising the isopropylbenzene is obtained, and a first gas stream comprising all of the propylene and more than 95% by weight of the propane, and also more than 90% by weight of the benzene, is obtained.

In this context, it is very especially preferable when the aforementioned first gas stream is sent to the second partial condensation under reflux with diversion, from which a second gas stream comprising at least 70% by weight each of propylene and propane from the first gas stream sent to the second partial condensation is obtained. In addition, a third liquid stream comprising at least 70% by weight of the benzene sent to the second partial condensation and a fourth liquid stream comprising at least 60% by weight of the isopropylbenzene sent to the second partial condensation are obtained from this second partial condensation.

The aforementioned first and second liquid streams are, according to the especially preferred execution of the third especially preferred development described here, sent to a distillation column. In this case, the first liquid stream is supplied in the stripping section of the column and the second liquid stream in the rectifying section of the column. Between the stripping and rectifying sections of the column, the reaction product (P) is removed.

The two partial condensations according to the above-detailed especially preferred embodiment of the invention are conducted here typically at a pressure in the range from 1 to 5 bar, preferably at a pressure of about 2 bar.

The first partial condensation is preferably conducted in the region of the scrubbing column at a temperature in the range from 150° C. to 190° C., and condensation heat exchanger and downstream collecting vessel at a temperature in the range from 140° C. to 160° C.

The second partial condensation is preferably conducted in the region of the scrubbing column at a temperature in the range from 100° C. to 150° C., and condensation heat exchanger and downstream collecting vessel at a temperature in the range from 60° C. to 110° C.

Within the above-described third especially preferred development of the process according to the invention, it is additionally likewise especially preferable that the reaction gas mixture comprises the reaction product (P) ethylbenzene, the uncondensable reaction gases (G) ethylene, propylene and ethane, the low boiler (L) benzene and the high boiler (H) diethylbenzene, and when this reaction gas mixture is sent to a first partial condensation under reflux with diversion, from which a first liquid stream comprising the entirety of the diethylbenzene and a proportion of not more than 10% by weight of the ethylbenzene is obtained, a second liquid stream comprising the ethylbenzene is obtained, and a first gas stream comprising more than 95% by weight each of the ethylene, propylene and ethane, and also more than 20% by weight of the benzene, is obtained.

In this context, it is very especially preferable when the aforementioned first gas stream is sent to the second partial condensation under reflux with diversion, from which a second gas stream comprising at least 90% by weight each of ethylene, propylene and ethane from the first gas stream sent to the second partial condensation is obtained.

In addition, a third liquid stream comprising at least 70% by weight of the benzene sent to the second partial condensation and a fourth liquid stream comprising at least 60% by weight of the ethylbenzene sent to the second partial condensation are obtained from this second partial condensation.

The aforementioned first and second liquid streams are, according to the especially preferred execution of the third especially preferred development described here, sent to a distillation column. In this case, the first liquid stream is supplied in the stripping section of the column and the second liquid stream in the rectifying section of the column. Between the stripping and rectifying sections of the column, the reaction product (P) is removed.

The two partial condensations according to the above-detailed especially preferred embodiment of the invention are conducted here typically at a pressure in the range from 1 to 3 bar, preferably at a pressure of about 1.1 bar.

The first partial condensation is preferably conducted in the region of the scrubbing column at a temperature in the range from 110° C. to 170° C., and condensation heat exchanger and downstream collecting vessel at a temperature in the range from 90° C. to 115° C.

The second partial condensation is preferably conducted in the region of the scrubbing column at a temperature in the range from 70° C. to 100° C., and condensation heat exchanger and downstream collecting vessel at a temperature in the range from 50° C. to 80° C.

Within the above-described fourth especially preferred development of the process according to the invention, it is especially preferable that the reaction gas mixture comprises a mixture of benzene and toluene as reaction product (P), ethylene, hydrogen and methane as uncondensable reaction gases (G), and water, ethylbenzene and styrene as high boilers (H), and when this reaction gas mixture is sent to a first partial condensation under reflux with phase separation, from which a first liquid stream comprising the entirety of the styrene and a proportion of at least 99% by weight of the ethylbenzene is obtained, a second liquid stream comprising at least 99% by weight of the water is obtained, and a first gas stream comprising more than 85% by weight of the reaction product is obtained.

In this context, it is very especially preferable when the aforementioned first gas stream is sent to the second partial condensation with phase separation, from which a second gas stream comprising at least 90% by weight each of hydrogen, methane and ethylene from the first gas stream sent to the second partial condensation is obtained.

In addition, a third liquid stream comprising at least 70% by weight of the reaction product (P) sent to the second partial condensation and a fourth liquid stream comprising the rest of the water in the gas stream sent to the second partial condensation are obtained from this second partial condensation.

The aforementioned first liquid stream is, according to the especially preferred execution of the fourth especially preferred development described here, sent to a distillation column. In this case, the first liquid stream is fed in between stripping section and rectifying section of the column. At the top of the column, the reaction product (P) is removed; at the bottom of the column, styrene is withdrawn.

Possible embodiments of the process according to the invention are illustrated in detail hereinafter with the aid of the figures and with reference to working examples, but without restricting the invention thereto.

The figures show,

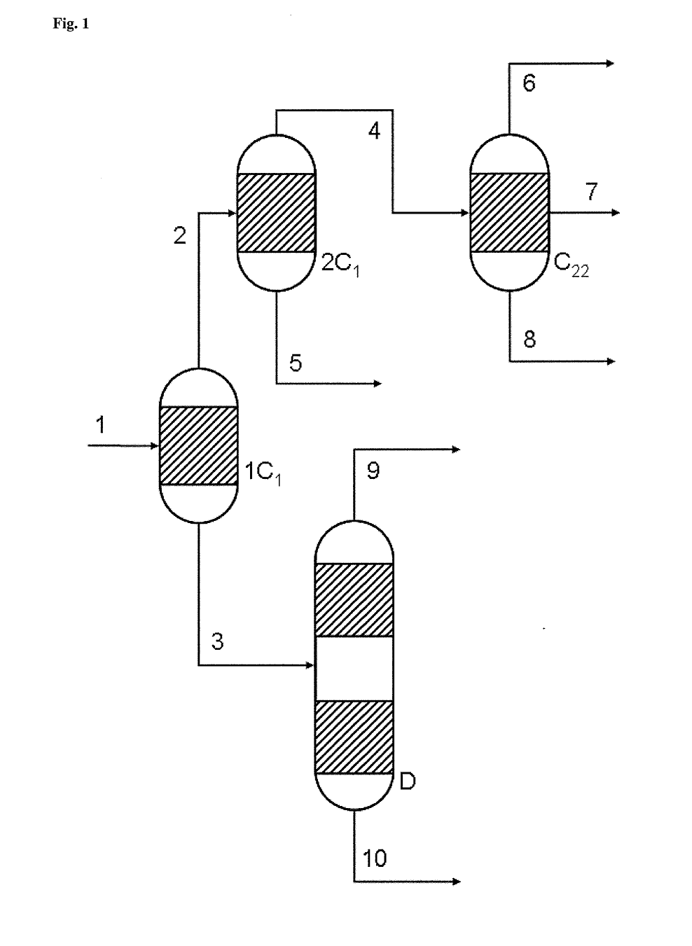

in FIG. 1, a process flow diagram according to Working Example 1,

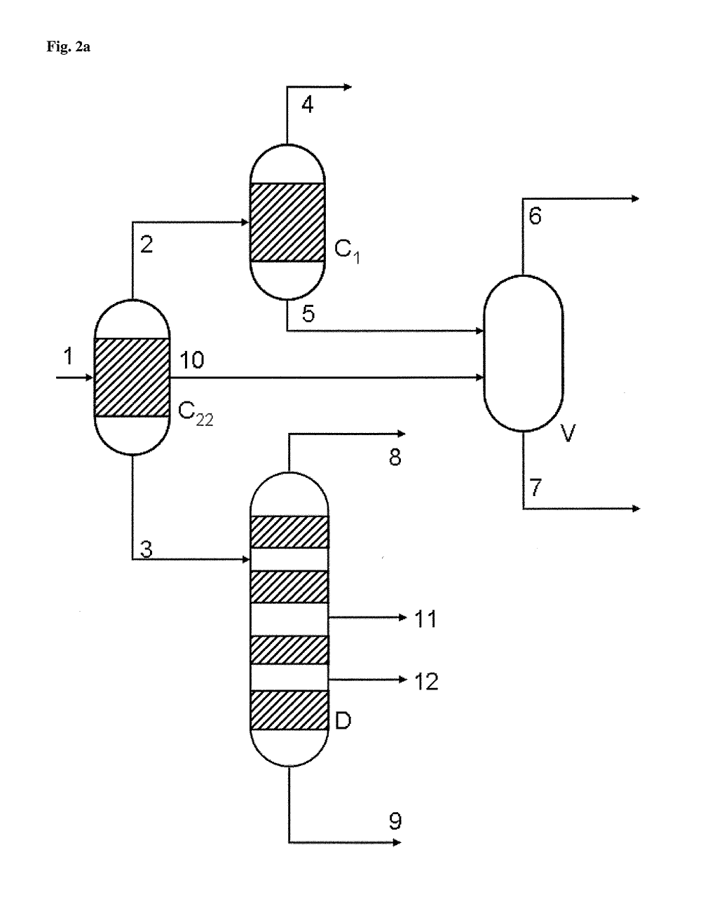

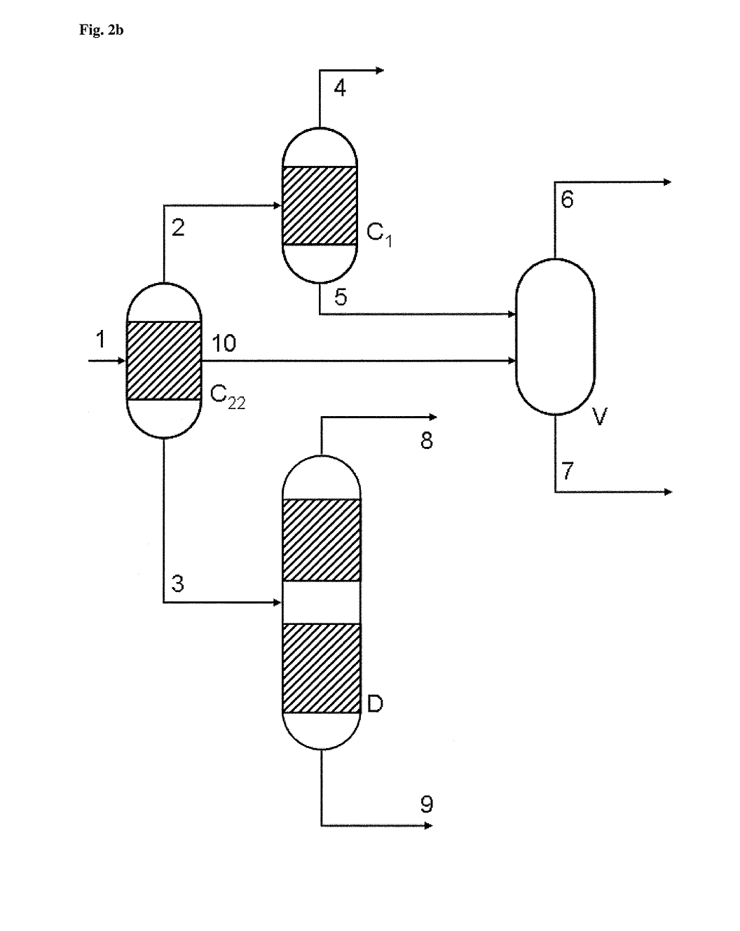

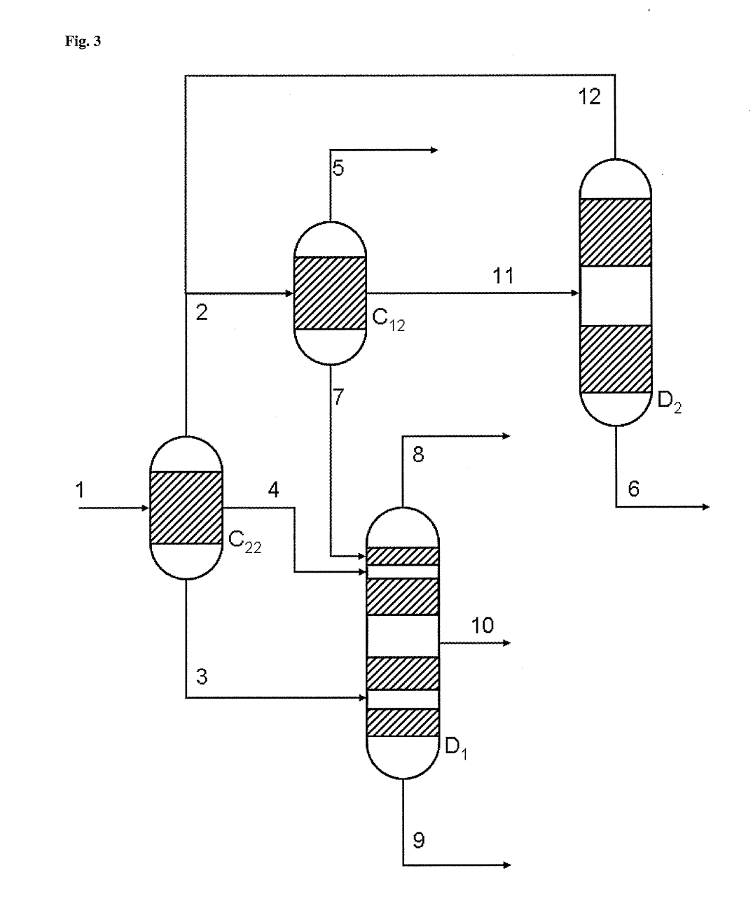

in FIG. 2, a process flow diagram according to Working Example 2 (FIG. 2a) and 3 (FIG. 2b),

in FIG. 3, a process flow diagram according to Working Example 4,

in FIG. 4, a process flow diagram according to Working Example 5 and 6 and,

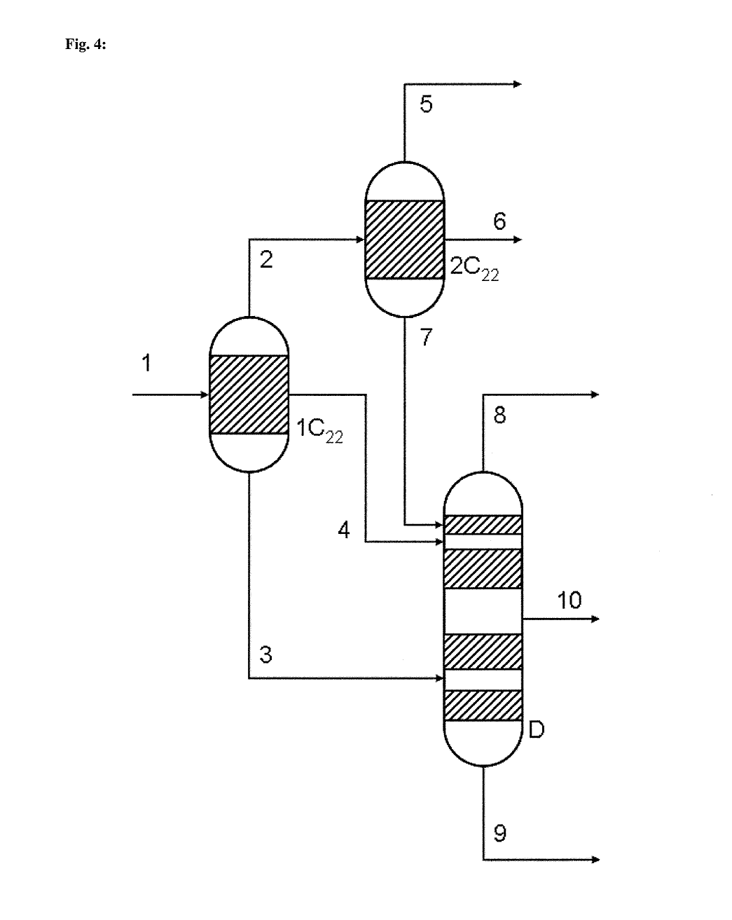

in FIG. 5, a process flow diagram according to Working Example 7.

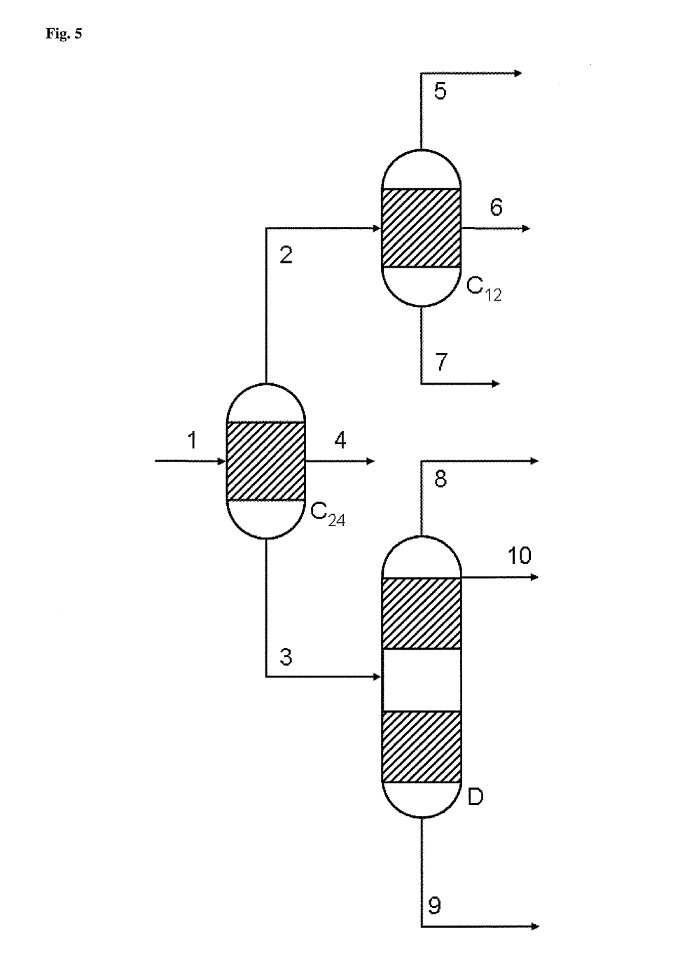

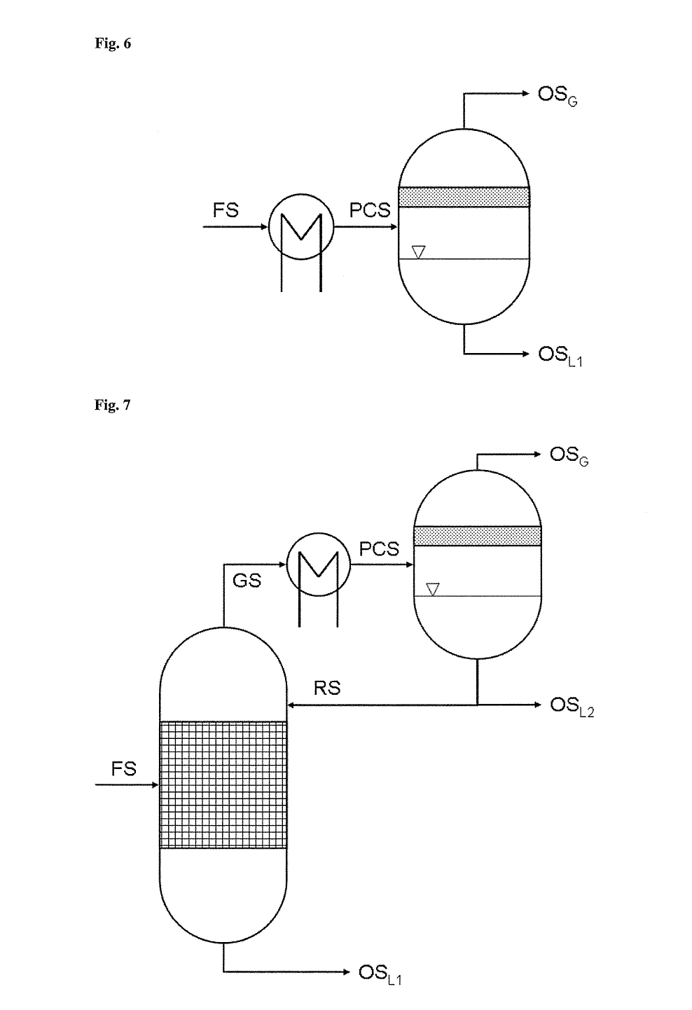

FIG. 6 shows one embodiment of a condenser for the inventive partial condensation. Such a condenser is supplied with a gas stream (FS) which is partly condensed in a condensation heat exchanger and leaves it again as stream (PCS) comprising at least one liquid phase and a gas phase. This stream (PCS) is separated in a collecting vessel with a droplet separator into a liquid stream (OS_{L1}) and a gas stream (OS_{G}). In FIGS. 1 and 2, a condenser possibly configured in this form is identified in each case with (C₁).

FIG. 7 shows a further embodiment of a condenser for the inventive partial condensation. This condenser allows partial condensation with reflux and diversion of a second liquid stream. Such a condenser is supplied with a gas stream (FS). This gas stream (FS) enters a scrubbing column containing random packings, where it is partly condensed with return of the condensate (RS). A first liquid stream (OS_{L1}) and a gas stream (GS) leave the scrubbing column. The gas stream is in turn partly condensed in a condensation heat exchanger and leaves it as stream (PCS) comprising at least one liquid phase and a gas phase. This stream (PCS) is separated in a collecting vessel with a droplet separator into a liquid stream and a gas stream (OS_{S}), the liquid stream being separated off and leaving the condenser in this proportion (OS_{L2}). The other portion of this liquid stream is fed back to the scrubbing column as condensate (RS). In FIGS. 1 to 4, a condenser possibly configured in this form is identified in each case with (C₂₂).

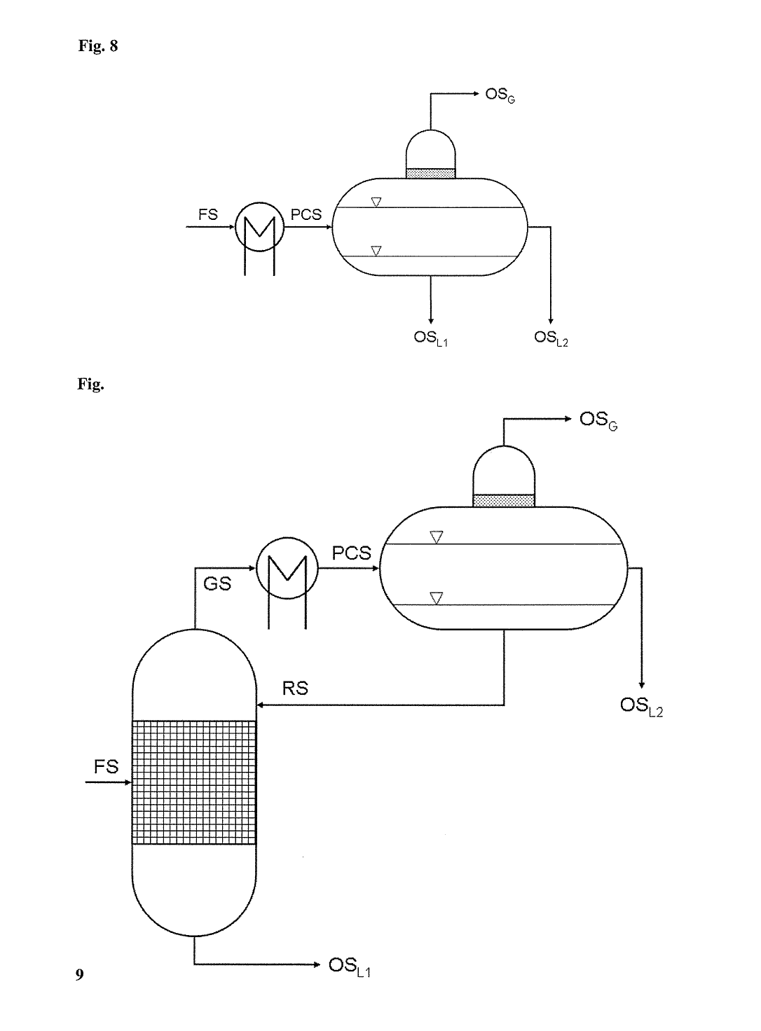

FIG. 8 shows another embodiment of a condenser for the inventive partial condensation. Such a condenser is supplied with a gas stream (FS) which is partly condensed in a condensation heat exchanger and leaves it again as stream (PCS) comprising two liquid phases and one gas phase. This stream (PCS) is separated in a collecting vessel with a droplet separator into a first liquid stream (OS_{L1}) and second liquid stream (OS_{L2}) and a gas stream (OS_{G}). The separation between first liquid stream (OS_{L1}) and second liquid stream (OS_{L2}) in the collecting vessel is effected by the gravitational phase separation of polar and nonpolar liquid phases of different density. In FIGS. 3 and 5, a condenser possibly configured in this form is identified in each case with (C₁₂).

FIG. 9 shows yet a further embodiment of a condenser for the inventive partial condensation. This is, more particularly, a particular combination of the features according to FIGS. 7 and 8. In such a condenser, a gas stream (FS) is sent to a scrubbing column as in FIG. 7, and is partly condensed with return of the condensate (RS). A first liquid stream (OS_{L1}) and a gas stream (GS) leave the scrubbing column, the latter being partly condensed in a condensation heat exchanger and leaving it again as stream (PCS) comprising two liquid phases and one gas phase. This stream (PCS) is separated in a collecting vessel with a droplet separator into a first liquid stream (RS) and second liquid stream (OS_{L2}) and a gas stream (OS_{G}). The separation between first liquid stream (RS) and second liquid stream (OS_{L2}) in the collecting vessel is effected by the gravitational phase separation of polar and nonpolar liquid phases of different density. The first liquid stream (RS) is the liquid stream comprising the phase of higher density. This is sent back to the scrubbing column. The second liquid stream (OS_{L2}) comprising the liquid phase of lower density is conducted out of the condenser. In FIG. 5, a condenser possibly configured in this form is identified with (C₂₄).

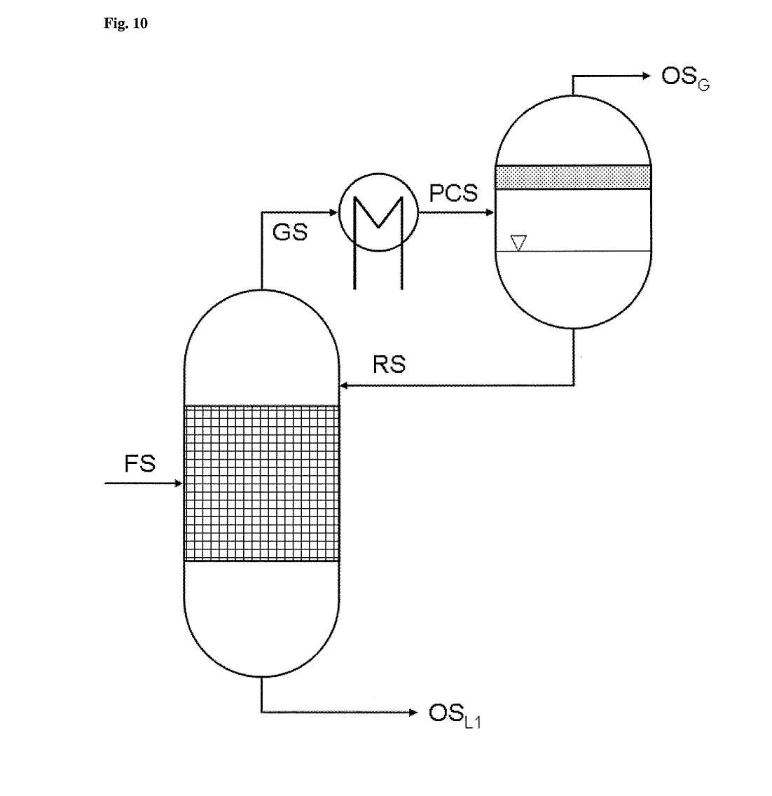

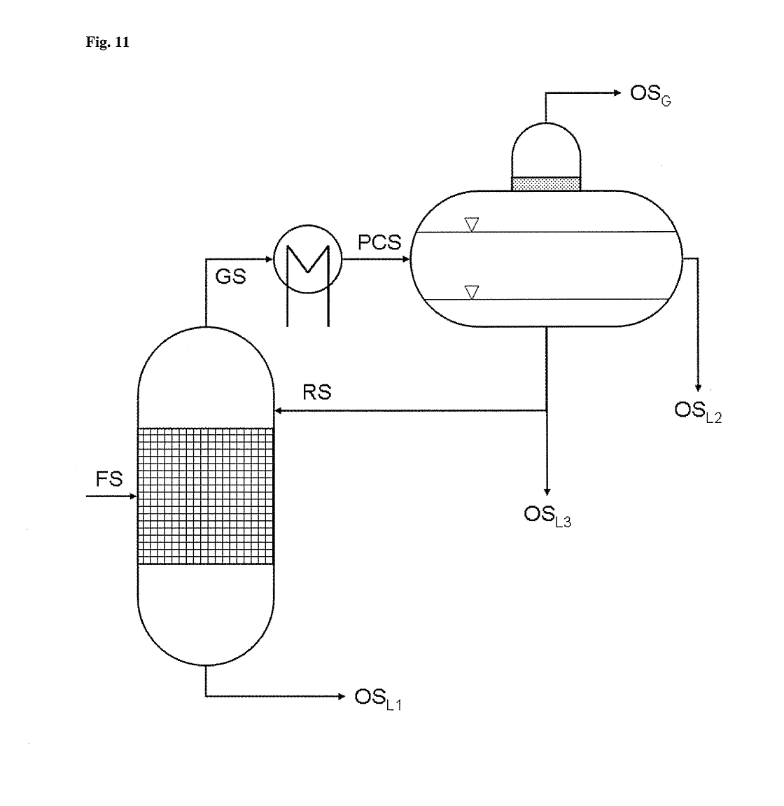

FIG. 10 shows a condenser of the design and mode of function as already described for FIG. 7, except that no diversion of a second liquid stream is provided.

FIG. 11 shows a condenser of the design and mode of function as already described for FIG. 9, except that a diversion of a third liquid stream (OS_{L3}) is provided.

### EXAMPLES

### Example 1

### Process According to First Especially Preferred Development Applied to Reaction Gas Mixtures Comprising the Reaction Product (P) Toluenediamine (TDA)

Proceeding from Example 1 of GB 832939, a reaction gas mixture according to Table 1 was obtained.

TABLE 1
State and composition of the reaction gas
mixture in stream (1) according to FIG. 1

| Parameter | Unit | Value |
|---|---|---|
| Temperature | ° C. | 220 |
| Pressure | bar | 3.15 |
| Mass flow rate . . . | [kg/h] | |
| of nitrogen (N₂) | [kg/h] | 29 083 |
| of hydrogen (H₂) | [kg/h] | 9387 |
| of water vapour (H₂O) | [kg/h] | 18 053 |
| of toluenediamine | [kg/h] | 23 481 |
| of impurities | [kg/h] | 24 |

This reaction gas mixture was subjected to a process as shown in the flow diagram in FIG. 1. For this purpose, the stream of reaction gas mixture (1) of the composition according to Table 1 was first partly condensed in a first condenser (1C₁) according to FIG. 6 at a temperature in the range from about 200° C. to 220° C., such that a first liquid stream (3) of the composition as reported in Table 2 and a first gas stream (2) of the composition as reported in Table 2 were obtained.

The first gas stream (2) was then subjected to a second partial condensation in a second condenser (2C₁) according to FIG. 6. From this condensation, a second gas stream (4) and a second liquid stream (5) were obtained. Both have compositions as reported in Table 2.

The second gas stream (4) was subjected to a third partial condensation in a third condenser (C₂₂) according to FIG. 7. This partial condensation was executed as a partial condensation under reflux and with diversion of a substream of the condensate, such that a third (7) and a fourth liquid stream (8) were obtained from the third partial condensation. In addition, a third gas stream (6) was obtained from the third partial condensation. The third and fourth liquid streams (7, 8), like the third gas stream (6) too, were again of the composition as reported in Table 2. The temperature in the scrubbing column was in the range from 86° C. to 160° C.; the temperature in the condensation heat exchanger and collecting vessel was in the range from 50° C. to 86° C. The above ranges arose owing to a temperature distribution which is established in the individual apparatuses.

The first liquid stream (3) was sent to a rectification column (D) between stripping section and rectifying section, from which a top stream (9) and a bottom stream (10), again of the composition according to Table 2, were withdrawn.

All aforementioned partial condensations were executed at pressures of about 3 bar. In other words, the pressures were not lowered beyond a degree as would arise in any case from the general pressure drop in the streams and/or condensers.

TABLE 2
Composition of the streams according to FIG. 1
in percent by mass of stream (1)

| | Boiling | | | | | | | | | |
|---|---|---|---|---|---|---|---|---|---|---|
| | point | Stream | | | | | | | | |
| Substance (type) | [° C.] | 2 | 3 | 4 | 5 | 6 | 7 | 8 | 9 | 10 |
| N₂ (G) | −195.8 | 100 | 0 | 100 | 0 | 100 | 0 | 0 | 100 | 0 |
| H₂ (G) | −252.8 | 100 | 0 | 100 | 0 | 100 | 0 | 0 | 100 | 0 |
| H₂O (L) | 100.0 | 100 | 0 | 100 | 0 | 24 | 75 | 0 | 100 | 0 |
| TDA (P) | 283.9 | 91 | 9 | 19 | 71 | 0 | 0 | 19 | 91 | 9 |
| impurities (H) | >300 | 0 | 100 | 0 | 0 | 0 | 0 | 0 | 0 | 100 |

This inventive process variant avoids the need for complete condensation and vaporization of the water and of the TDA present in the reaction gas mixture (1), which leads to significant energy savings, by using the enthalpy of the reaction gas mixture to maximum effect in the aforementioned partial condensations for separation of the components.

### Example 2

### Process According to First and Second Preferred Developments Comprehensively Applied to Reaction Gas Mixtures Comprising the Reaction Product (P) Methanol

A reaction gas mixture according to Table 3 was subjected to the process described below.

TABLE 3
State and composition of the reaction gas
mixture in stream (1) according to Ex. 2

| Parameter | Unit | Value |
|---|---|---|
| Temperature | ° C. | 160 |
| Pressure | bar | 75 |
| Mass flow rate . . . | [kg/h] | |
| of carbon monoxide (CO) | [kg/h] | 9241 |
| of carbon dioxide (CO₂) | [kg/h] | 25 788 |
| of hydrogen (H₂) | [kg/h] | 6123 |
| of nitrogen (N₂) | [kg/h] | 2858 |
| of methane (CH₄) | [kg/h] | 13 605 |
| of dimethyl ether | [kg/h] | 253 |
| of methanol | [kg/h] | 9285 |
| of ethanol | [kg/h] | 14 |
| of water (H₂O) | [kg/h] | 2285 |

This reaction gas mixture was subjected to a process as shown in the flow diagram in FIG. 2a. For this purpose, the stream of reaction gas mixture (1) of the composition according to Table 3 was first subjected to a first partial condensation in a first condenser (C₂₂) according to FIG. 7.

This first partial condensation was executed under reflux and with diversion of a portion of the condensate, such that a first liquid stream (3) and a second liquid stream (10) were obtained.

The first condenser (C₂₂) was operated at a temperature in the range of 128° C. to 160° C. in the scrubbing column and in the range from 78° C. to 116° C. in the condensation heat exchanger and collecting vessel.

From this first partial condensation, a first gas stream (2) was also obtained, which was sent to a second partial condensation in a second condenser (C₁) according to FIG. 6. This second partial condensation was executed at temperatures in the range from 40° C. to 78° C., and a second gas stream (4) and a third liquid stream (5) were obtained therefrom. The liquid streams (5) and (10) were fed together to a stripper (V), from which a gas stream (6) and a liquid stream (7) were obtained. The liquid stream (3) was fed to a rectification column (D) between stripping section and rectifying section, and pure methanol was withdrawn therefrom in the stripping section.

TABLE 4
Composition of the streams according to FIG. 2a in percent by mass of stream (1)

| | Boiling | | | | | | | | | | | |
|---|---|---|---|---|---|---|---|---|---|---|---|---|
| | point | Stream | | | | | | | | | | |
| Substance (type) | [° C.] | 2 | 3 | 4 | 5 | 6 | 7 | 8 | 9 | 10 | 11 | 12 |
| H₂ (G) | −252.8 | 100 | 0 | 100 | 0 | 0 | 0 | 0 | 0 | 0 | 0 | 0 |
| N₂ (G) | −195.8 | 100 | 0 | 100 | 0 | 0 | 0 | 0 | 0 | 0 | 0 | 0 |
| CO (G) | −191.5 | 100 | 0 | 100 | 0 | 0 | 0 | 0 | 0 | 0 | 0 | 0 |
| CH₄ (G) | −161.5 | 99 | 0 | 99 | 0 | 1 | 0 | 0 | 0 | 0 | 0 | 0 |
| CO₂ (G) | −78.5 | 98 | 1 | 97 | 2 | 2 | 0 | 1 | 0 | 1 | 0 | 0 |
| dimethyl ether (L) | −24.9 | 95 | 4 | 92 | 3 | 4 | 0 | 1 | 0 | 1 | 0 | 0 |
| methanol (P) | 64.7 | 36 | 26 | 7 | 29 | 4 | 63 | 0 | 0 | 38 | 25 | 1 |
| ethanol (H) | 78.3 | 29 | 25 | 3 | 26 | 3 | 69 | 0 | 0 | 46 | 2 | 23 |
| water (H) | 100.0 | 0 | 100 | 0 | 0 | 0 | 0 | 0 | 100 | 0 | 0 | 0 |

The respective streams (2-12) are of the compositions according to Table 4. Except for the aforementioned stripper (V) and the rectification column (D), the process was executed at pressures of about 50-100 bar. The stripper (V) and the rectification column (D) were operated at a pressure below 50 bar, specifically at about ambient pressure (1013 hPa). In other words, the pressures were not lowered beyond a degree as would arise in any case from the general pressure drop in the streams and/or condensers.

This inventive process variant avoids the need for complete condensation and vaporization of the methanol present in the reaction gas mixture (1), which leads to significant energy savings, by using the enthalpy of the reaction gas mixture to maximum effect in the aforementioned partial condensations for separation of the components.

### Example 3

### Process According to First and Second Preferred Developments Comprehensively Applied to Reaction Gas Mixtures Comprising the Reaction Product (P) Benzene

A reaction gas mixture according to Table 5 was subjected to the process described below.

TABLE 5
State and composition of the reaction gas
mixture in stream (1) according to Ex. 3

| Parameter | Unit | Value |
|---|---|---|
| Temperature | ° C. | 220 |
| Pressure | bar | 35 |
| Mass flow rate . . . | [kg/h] | |
| of hydrogen (H₂) | [kg/h] | 1270 |
| of nitrogen (N₂) | [kg/h] | 4176 |
| of methane (CH₄) | [kg/h] | 18 192 |
| of benzene | [kg/h] | 11 810 |
| of toluene | [kg/h] | 3289 |

This reaction gas mixture was likewise subjected to a process as shown in the flow diagram in FIG. 2b. For this purpose, the stream of reaction gas mixture (1) of the composition according to Table 5 was first subjected to a first partial condensation in a first condenser (C₂₂) according to FIG. 7.

This first partial condensation was executed under reflux and with diversion of a portion of the condensate, such that a first liquid stream (3) and a second liquid stream (10) were obtained.

The first condenser (C₂₂) was operated at a temperature in the range of 146° C. to 220° C. in the scrubbing column and in the range from 75° C. to 146° C. in the condensation heat exchanger and collecting vessel.

From this first partial condensation, a first gas stream (2) was also obtained, which was sent to a second partial condensation in a second condenser (C₁) according to FIG. 6. This second partial condensation was executed at temperatures in the range from 50° C. to 75° C., and a second gas stream (4) and a third liquid stream (5) were obtained therefrom. The liquid streams (5) and (10) were fed together to a stripper (V), from which a gas stream (6) and a liquid stream (7) were obtained. The liquid stream (3) was fed to a rectification column (D) between stripping section and rectifying section, and pure methanol was withdrawn therefrom in the stripping section.

The respective streams (2-10) are of the compositions according to Table 4. Except for the aforementioned stripper, the process was executed at pressures of about 75 bar. In other words, the pressures were not lowered beyond a degree as would arise in any case from the general pressure drop in the streams and/or condensers.

TABLE 6
Composition of the streams according to FIG. 2b
in percent by mass of stream (1)

| | Boiling | | | | | | | | | |
|---|---|---|---|---|---|---|---|---|---|---|
| Substance | point | Stream | | | | | | | | |
| (type) | [° C.] | 2 | 3 | 4 | 5 | 6 | 7 | 8 | 9 | 10 |
| H₂ (G) | −252.8 | 100 | 0 | 100 | 0 | 0 | 0 | 0 | 0 | 0 |
| N₂ (G) | −195.8 | 100 | 0 | 100 | 0 | 0 | 0 | 0 | 0 | 0 |
| CH₄ (G) | −161.5 | 100 | 0 | 100 | 0 | 0 | 0 | 0 | 0 | 0 |
| benzene | 80 | 31 | 40 | 13 | 18 | 1 | 47 | 40 | 0 | 47 |
| (P) | | | | | | | | | | |
| toluene | 110 | 0 | 100 | 0 | 0 | 0 | 0 | 0 | 100 | 0 |
| (H) | | | | | | | | | | |

This inventive process variant avoids the need for complete condensation and vaporization of the benzene present in the reaction gas mixture (1), which leads to significant energy savings, by using the enthalpy of the reaction gas mixture to maximum effect in the aforementioned partial condensations for separation of the components.

### Example 4

### Process According to Second Especially Preferred Development Applied to Reaction Gas Mixtures Comprising a Mixture of Aniline and Phenol as Reaction Product (P)

A reaction gas mixture according to Table 7 was subjected to the process described below.

TABLE 7
State and composition of the reaction gas
mixture in stream (1) according to FIG. 3

| Parameter | Unit | Value |
|---|---|---|
| Temperature | ° C. | 147 |
| Pressure | bar | 5.1 |
| Mass flow rate . . . | [kg/h] | |
| of hydrogen (H₂) | [kg/h] | 2922 |
| of nitrogen (N₂) | [kg/h] | 1526 |
| of ammonia (NH₃) | [kg/h] | 41 |
| of benzene | [kg/h] | 84 |
| of water | [kg/h] | 4661 |
| of aniline | [kg/h] | 7443 |
| of phenol | [kg/h] | 6 |
| of diphenylamine | [kg/h] | 10 |

This reaction gas mixture was subjected to a process as shown in the flow diagram in FIG. 3. For this purpose, the stream of reaction gas mixture (1) of the composition according to Table 7 was first subjected to a first partial condensation in a first condenser (C₂₂) according to FIG. 7.

This first partial condensation was executed under reflux and with diversion of a portion of the condensate, such that a first liquid stream (3) and a second liquid stream (4) were obtained.

The first condenser (C₂₂) was operated at a temperature in the range of 145° C. to 147° C. in the scrubbing column and in the range from 118° C. to 145° C. in the condensation heat exchanger and collecting vessel.

From this first partial condensation, a first gas stream (2) was also obtained, which was sent to a second partial condensation with phase separation in a second condenser (C₁₂) according to FIG. 8 together with the top stream (12) from a second rectification column (D₂).

This second partial condensation was executed at temperatures in the range from 60° C. to 118° C., and a second gas stream (5) and a third (7) and fourth liquid stream (11) were obtained therefrom. The third liquid stream (7) contained essentially the nonpolar components and the fourth liquid stream (11) the polar components. The fourth liquid stream (11) is fed to a second rectification column (D₂), from which a further liquid stream (6) comprising essentially water and a top stream (12) comprising essentially ammonia and water were obtained.

TABLE 8
Composition of the streams according to FIG. 3 in percent by mass of stream (1)

| | Boiling | | | | | | | | | | | |
|---|---|---|---|---|---|---|---|---|---|---|---|---|
| | point | Stream | | | | | | | | | | |
| Substance (type) | [° C.] | 2 | 3 | 4 | 5 | 6 | 7 | 8 | 9 | 10 | 11 | 12 |
| H₂ (G) | −252.8 | 100 | 0 | 0 | 100 | 0 | 0 | 0 | 0 | 0 | 0 | 0 |
| N₂ (G) | −195.8 | 100 | 0 | 0 | 100 | 0 | 0 | 0 | 0 | 0 | 0 | 0 |
| NH₃ (G) | −33.4 | 100 | 0 | 0 | 81 | 0 | 18 | 19 | 0 | 0 | 122 | 122 |
| benzene (L) | 80.1 | 97 | 1 | 3 | 94 | 0 | 2 | 6 | 0 | 0 | 0 | 0 |
| water (L) | 100.0 | 98 | 0 | 2 | 20 | 72 | 4 | 8 | 0 | 0 | 106 | 34 |
| aniline (P) | 184.0 | 35 | 20 | 45 | 2 | 0 | 33 | 2 | 0 | 96 | 2 | 2 |
| phenol (P) | 181.8 | 20 | 29 | 51 | 1 | 1 | 19 | 1 | 0 | 98 | 1 | 1 |
| diphenylamine (H) | 302.0 | 0 | 100 | 0 | 0 | 0 | 0 | 0 | 100 | 0 | 0 | 0 |

The liquid streams (3), (4) and (7) were fed to a first rectification column (D) separately from one another at different points. The liquid streams (4) and (7) were fed to the rectifying section and the liquid stream (3) to the stripping section of the rectification column (D). It was found that it is unimportant whether streams (4) and (7) were fed separately or together to the first rectification column (D₁). What was essential was merely that they were fed to the first rectification column (D₁) in the rectifying section, while stream (3) was fed to the stripping section. Between the stripping and rectifying sections, a pure mixture (10) of aniline and phenol was withdrawn.

The respective streams (2-11) are of the compositions according to Table 8. Except for the rectification columns (D₁, D₂), the process was executed at pressures of about 5 bar. The rectification columns (D₁, D₂) were operated at absolute pressures in the range from 100 to 600 mbar. In other words, the pressures were essentially not lowered beyond a degree as would arise in any case from the general pressure drop in the streams and/or condensers.

This inventive process variant avoids the need for complete condensation and vaporization of the aniline present in the reaction gas mixture (1), which leads to significant energy savings, by using the enthalpy of the reaction gas mixture to maximum effect in the aforementioned partial condensations for separation of the components.

### Example 5

### Process According to Third Especially Preferred Development Applied to Reaction Gas Mixtures Comprising the Reaction Product (P) Isopropylbenzene

A reaction gas mixture according to Table 9 was subjected to the process described below.

TABLE 9
State and composition of the reaction gas
mixture in stream (1) according to Ex. 5

| Parameter | Unit | Value |
|---|---|---|
| Temperature | ° C. | 190 |
| Pressure | bar | 2.5 |
| Mass flow rate . . . | [kg/h] | |
| of propylene (H₂) | [kg/h] | 373 |
| of propane (N₂) | [kg/h] | 355 |
| of benzene (NH₃) | [kg/h] | 8511 |
| of isopropylbenzene | [kg/h] | 11 345 |
| of diisopropylbenzene | [kg/h] | 453 |

This reaction gas mixture was subjected to a process as shown in the flow diagram in FIG. 4. For this purpose, the stream of reaction gas mixture (1) of the composition according to Table 9 was first subjected to a first partial condensation in a first condenser (1C₂₂) according to FIG. 7.

This first partial condensation was executed under reflux and with diversion of a portion of the condensate, such that a first liquid stream (3) and a second liquid stream (4) were obtained.

The first condenser (1C₂₂) was operated at a temperature in the range of 156° C. to 190° C. in the scrubbing column and in the range from 145° C. to 156° C. in the condensation heat exchanger and collecting vessel.

From this first partial condensation, a first gas stream (2) was also obtained, which was sent to a second partial condensation under reflux with diversion in a second condenser (2C₂₂) according to FIG. 7. From this second partial condensation, a third (6) and a fourth (7) liquid stream were obtained, as was a second gas stream (5).

The second condenser (2C₂₂) was operated at a temperature in the range of 102° C. to 145° C. in the scrubbing column and in the range from 65° C. to 102° C. in the condensation heat exchanger and collecting vessel.

TABLE 10
Composition of the streams according to FIG. 4 in
percent by mass of stream (1) in Ex. 5

| | Boiling | | | | | | | | | |
|---|---|---|---|---|---|---|---|---|---|---|
| | point | Stream | | | | | | | | |
| Substance (type) | [° C.] | 2 | 3 | 4 | 5 | 6 | 7 | 8 | 9 | 10 |
| propylene (G) | −47.7 | 100 | 0 | 0 | 81 | 18 | 1 | 1 | 0 | 0 |
| propane (G) | −42 | 99 | 0 | 1 | 71 | 28 | 1 | 2 | 0 | 0 |
| benzene (L) | 80.1 | 91 | 1 | 8 | 5 | 72 | 14 | 22 | 0 | 1 |
| isopropylbenzene (P) | 152.4 | 59 | 9 | 31 | 0 | 1 | 59 | 1 | 1 | 98 |
| diisopropylbenzene (H) | 210.5 | 0 | 100 | 0 | 0 | 0 | 0 | 0 | 100 | 0 |

The liquid streams (3), (4) and (7) were fed together to a rectification column (D). The liquid streams (4) and (7) were fed to the rectifying section and the liquid stream (3) to the stripping section of the rectification column. Between stripping section and rectifying section, pure isopropylbenzene (10) was withdrawn. In addition, a top stream (8) and bottom stream (9) were withdrawn from the column.

The respective streams (2-10) are of the compositions according to Table 10. The process was executed at pressures of about 2 bar. In other words, the pressures were not lowered beyond a degree as would arise in any case from the general pressure drop in the streams and/or condensers.

This inventive process variant avoids the need for complete condensation and vaporization of the benzene and isopropylbenzene present in the reaction gas mixture (1), which leads to significant energy savings, by using the enthalpy of the reaction gas mixture to maximum effect in the aforementioned partial condensations for separation of the components.

### Example 6

### Process According to Third Especially Preferred Development Applied to Reaction Gas Mixtures Comprising the Reaction Product (P) Ethylbenzene

A reaction gas mixture according to Table 11 was subjected to the process described below.

TABLE 11
State and composition of the reaction gas
mixture in stream (1) according to Ex. 6

| Parameter | Unit | Value |
|---|---|---|
| Temperature | ° C. | 170 |
| Pressure | bar | 1.1 |
| Mass flow rate . . . | [kg/h] | |
| of ethylene | [kg/h] | 15 |
| of ethane | [kg/h] | 210 |
| of propylene | [kg/h] | 84 |
| of benzene | [kg/h] | 13 889 |
| of ethylbenzene | [kg/h] | 9789 |
| of diethylbenzene | [kg/h] | 1382 |

This reaction gas mixture was likewise subjected to a process as shown in the flow diagram in FIG. 4. For this purpose, the stream of reaction gas mixture (1) of the composition according to Table 11 was first subjected to a first partial condensation in a first condenser (1C₂₂) according to FIG. 7.

This first partial condensation was executed under reflux and with diversion of a portion of the condensate, such that a first liquid stream (3) and a second liquid stream (4) were obtained.

The first condenser (1C₂₂) was operated at a temperature in the range of 111° C. to 170° C. in the scrubbing column and in the range from 90° C. to 111° C. in the condensation heat exchanger and collecting vessel.

From this first partial condensation, a first gas stream (2) was also obtained, which was sent to a second partial condensation under reflux with diversion in a second condenser (2C₂₂) according to FIG. 7. From this second partial condensation, a third (6) and a fourth (7) liquid stream were obtained, as was a second gas stream (5).

The second condenser (2C₂₂) was operated at a temperature in the range of 78° C. to 90° C. in the scrubbing column and in the range from 55° C. to 78° C. in the condensation heat exchanger and collecting vessel.

TABLE 12
Composition of the streams according to FIG. 4 in
percent by mass of stream (1) in Ex. 6

| | Boiling | | | | | | | | | |
|---|---|---|---|---|---|---|---|---|---|---|
| Substance | point | Stream | | | | | | | | |
| (type) | [° C.] | 2 | 3 | 4 | 5 | 6 | 7 | 8 | 9 | 10 |
| ethylene (G) | −103.7 | 98 | 0 | 2 | 96 | 2 | 0 | 2 | 0 | 0 |
| ethane (G) | −88.6 | 98 | 0 | 2 | 94 | 3 | 0 | 3 | 0 | 0 |
| propylene (G) | −47.7 | 95 | 0 | 5 | 88 | 6 | 1 | 6 | 0 | 0 |
| benzene (L) | 80.1 | 29 | 4 | 67 | 3 | 18 | 8 | 79 | 0 | 0 |
| ethylbenzene | 136.2 | 6 | 21 | 73 | 0 | 0 | 6 | 2 | 1 | 97 |
| (P) | | | | | | | | | | |
| diethylbenzene | 183.5 | 0 | 100 | 0 | 0 | 0 | 0 | 0 | 100 | 0 |
| (H) | | | | | | | | | | |

The liquid streams (3), (4) and (7) were fed together to a rectification column (D). The liquid streams (4) and (7) were fed to the rectifying section and the liquid stream (3) to the stripping section of the rectification column. Between stripping section and rectifying section, pure isopropylbenzene (10) was withdrawn. In addition, a top stream (8) and bottom stream (9) were withdrawn from the column.

The respective streams (2-10) are of the compositions according to Table 12. The process was executed at pressures of about 1.1 bar. In other words, the pressures were not lowered beyond a degree as would arise in any case from the general pressure drop in the streams and/or condensers.

This inventive process variant avoids the need for complete condensation and vaporization of the benzene and ethylbenzene present in the reaction gas mixture (1), which leads to significant energy savings, by using the enthalpy of the reaction gas mixture to maximum effect in the aforementioned partial condensations for separation of the components.

### Example 7

### Process According to Fourth Especially Preferred Development Applied to Reaction Gas Mixtures Comprising a Mixture of Benzene and Toluene as Reaction Product (P)

A reaction gas mixture according to Table 13 was subjected to the process described below.

TABLE 13
State and composition of the reaction gas
mixture in stream (1) according to FIG. 5

| Parameter | Unit | Value |
|---|---|---|
| Temperature | ° C. | 250 |
| Pressure | bar | 1.5 |
| Mass flow rate . . . | [kg/h] | |
| of hydrogen (H₂) | [kg/h] | 178 |
| of methane (CH₄) | [kg/h] | 499 |
| of ethylene | [kg/h] | 721 |
| of benzene | [kg/h] | 2148 |
| of toluene | [kg/h] | 3068 |
| of water | [kg/h] | 144 122 |
| of ethylbenzene | [kg/h] | 35 710 |
| of styrene | [kg/h] | 12 568 |

This reaction gas mixture was subjected to a process as shown in the flow diagram in FIG. 5. For this purpose, the stream of reaction gas mixture (1) of the composition according to Table 13 was first subjected to a first partial condensation in a first condenser (C₂₄) according to FIG. 9.

The first partial condensation was executed under reflux and with phase separation, such that a first liquid stream (3) and a second liquid stream (4) were obtained. The first liquid stream (3) contained essentially the nonpolar components, while the second liquid stream (4) comprised essentially the polar components, i.e. the water together with entrained gas residues of methane and ethylene.

The first condenser (C₂₄) was operated at a temperature in the range of 72° C. to 250° C. in the scrubbing column and in the range from 65° C. to 72° C. in the condensation heat exchanger and collecting vessel.

From this first partial condensation, a first gas stream (2) was also obtained, which was sent to a second partial condensation with phase separation in a second condenser (C₁₂) according to FIG. 8.

TABLE 14
Composition of the streams according to
FIG. 5 in percent by mass of stream (1)

| | Boiling | | | | | | | | | |
|---|---|---|---|---|---|---|---|---|---|---|
| | point | Stream | | | | | | | | |
| Substance (type) | [° C.] | 2 | 3 | 4 | 5 | 6 | 7 | 8 | 9 | 10 |
| hydrogen (G) | −252 | 100 | 0 | 0 | 100 | 0 | 0 | 0 | 0 | 0 |
| methane (G) | −161.5 | 80 | 0 | 20 | 79 | 1 | 0 | 0 | 0 | 0 |
| ethylene (G) | −130.7 | 70 | 0 | 30 | 69 | 1 | 0 | 0 | 0 | 0 |
| benzene (P) | 80 | 93 | 3 | 4 | 35 | 58 | 0 | 3 | 0 | 0 |
| toluene (P) | 110 | 83 | 15 | 3 | 13 | 70 | 0 | 15 | 0 | 0 |
| water (H) | 100 | 1 | 0 | 99 | 0 | 0 | 1 | 0 | 0 | 0 |
| ethylbenzene (H) | 136 | 1 | 99 | 0 | 0 | 1 | 0 | 0 | 0 | 99 |
| styrene (H) | 145 | 0 | 100 | 0 | 0 | 0 | 0 | 0 | 100 | 0 |

This second partial condensation was executed at temperatures in the range from 35° C. to 65° C., and a second gas stream (5) and a third (6) and fourth liquid stream (7) were obtained therefrom. The third liquid stream (6) contained essentially the nonpolar components and the fourth liquid stream (7) the polar components in the form of pure water.

The liquid stream (3) was fed to a rectification column (D) between stripping section and rectifying section. At the top of the column (D), pure reaction product (8) was withdrawn in the form of a mixture of benzene and toluene. Just below the top of the column, pure ethylbenzene was withdrawn in a further stream (10), and pure styrene (9) was withdrawn at the bottom of the column.

The respective streams (2-10) are of the compositions according to Table 14. The process was executed at pressures of about 1.5 bar. In other words, the pressures were not lowered beyond a degree as would arise in any case from the general pressure drop in the streams and/or condensers.

This inventive process variant avoids the need for complete condensation and vaporization of benzene and toluene and additionally allows the further separation of the benzene and toluene components without a further distillation column. This leads to significant energy savings by using the enthalpy of the reaction gas mixture to maximum effect in the aforementioned partial condensations for separation of the components.

1. A process for simplified separation of a reaction product from a reaction gas mixture at a temperature above room temperature, said process involving use of at least one low boiler and/or uncondensable reaction gas and at least one high boiler, said process comprising at least one distillation and/or rectification, that is preceded by subjecting the reaction gas mixture to at least two partial condensations in which, in at least one
a) first partial condensation, at least 80% by weight of the high boiler supplied to said first partial condensation are condensed, and at least one liquid stream comprising said high boiler and any reaction product, and a gas stream comprising the reaction product, are thus obtained therefrom, and, in a
b) second partial condensation, at most 5% by weight of the low boiler supplied to said second partial condensation and/or uncondensable reaction gas are condensed, and at least one liquid stream comprising supplied reaction product, and a gas stream comprising the supplied low boiler and/or uncondensable reaction gas, are thus obtained therefrom,
by
1) supplying at least one liquid stream comprising reaction product to the at least one distillation and/or rectification at a point in a distillation and/or rectification column above a point from which the reaction product is drawn off from said distillation and/or rectification column, and/or
2) supplying at least one liquid stream comprising at least one high boiler to the at least one distillation and/or rectification at a point in the distillation and/or rectification column below a point from which reaction product is drawn off from said distillation and/or rectification column.

2. Process according to claim 1, wherein the first partial condensation according to a) and the second partial condensation according to b) are executed in direct succession, and a gas stream from the first partial condensation is supplied immediately to the second partial condensation.

3. Process according to claim 2, wherein the gas stream obtained from the second partial condensation, also comprising said reaction product, is sent to a third partial condensation in which at least 80% by weight of the reaction product supplied to a third partial condensation is condensed.

4. Process according to claim 1 wherein at least one of the partial condensations is executed with reflux of condensate thereof.

5. Process according to claim 4 wherein a portion of recycled condensate is removed and conducted out of said partial condensation as a further liquid stream.

6. Process according to claim 1 wherein at least one of the partial condensations also comprises a phase separation between two liquid phases which are not completely miscible with one another under conditions of partial condensation.

7. Process according to claim 1, wherein the reaction gas mixture comprises the reaction product toluenediamine, the uncondensable reaction gases hydrogen and nitrogen, the low boiler water and high boiler having a boiling point of more than 300° C. at ambient pressure.

8. Process according to claim 1, wherein the reaction gas mixture comprises the reaction product methanol, the uncondensable reaction gases carbon monoxide, carbon dioxide, hydrogen, nitrogen and methane, the low boiler dimethyl ether and the high boilers water and ethanol, or the reaction gas mixture comprises the reaction product benzene, the uncondensable reaction gases hydrogen, nitrogen and methane, and the high boiler toluene.

9. Process according to claim 1, wherein the reaction gas mixture comprises a mixture of benzene and toluene as said reaction product, ethylene, hydrogen and methane as said uncondensable reaction gases, and water, ethylbenzene and styrene as said high boilers.

10. Process according to claim 1, wherein the reaction gas mixture comprises the reaction product isopropylbenzene, the uncondensable reaction gases propylene and propane, the low boiler benzene and the high boiler diisopropylbenzene or the reaction product (P) ethylbenzene, the uncondensable reaction gases ethylene, propylene and ethane, the low boiler benzene and the high boiler diethylbenzene.
